(19) United States
(12) Patent Application Publication
(10) Pub. No.: US 2011/0009680 A1
(43) Pub. Date: Jan. 13, 2011

(54) Molecular Sieve Composition and Method of Making and Using the Same

(76) Inventor: Sebastien Kremer, Woluwe-Saint-Lambert (BE)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 12/920,532

(22) PCT Filed: Feb. 13, 2009
(86) PCT No.: PCT/US09/34078

§ 371 Date: Sep. 1, 2010

Related U.S. Application Data
(60) Provisional application No. 61/040,935, filed on Mar. 31, 2008.

(51) Int. Cl. C01B 39/02; C01B 39/04; C07C 5/02; C07C 5/32; C07C 1/00; C07C 5/31; C07C 15/02; C07C 4/06; C10G 73/02; C07C 5/25; C07C 5/27; C07C 2/02; C08F 4/00; C07C 2/58; C07C 15/12

(52) U.S. Cl. 585/250; 423/700; 423/704; 585/654; 585/639; 585/365; 585/400; 585/653; 208/28; 585/666; 585/739; 585/520; 585/510; 526/194; 585/722; 585/375; 585/475

(57) ABSTRACT

This disclosure relates to a crystalline molecular sieve comprising silicalite-1 having substantially hexagonal column morphology of at least 90% and having less than 20% crystal twinning as measured by SEM. This disclosure also relates to a method of making the crystalline molecular sieve of this disclosure, the method comprises:
(a) providing a mixture comprising at least one source of at least one tetravalent element (Y), at least one source of hydroxide ion, at least one directing-agent (R), water, the mixture having the following molar composition:
   H₂O/Y=10 to 1000
   OH⁻/Y=0.41 to 0.74
   R/Y=0.001 to 2
      wherein R comprises at least one of TPAOH, TPACl, TPABr, TPAI, and TPAF, wherein OH⁻/Y is not corrected for trivalent ion;
(b) submitting the mixture at crystallization conditions to form a product comprising the crystalline molecular sieve, wherein the crystallization conditions comprise a temperature in the range of from 100° C. to 250° C., a crystallization time from about 1 hour to 200 hours; a heating rate in the range from at least 20° C./h, and a stirring speed at least 10 RPM; and
(c) recovering the crystalline molecular sieve.

### FIELD OF THE INVENTION

The present disclosure relates to a crystalline molecular sieve comprising silicalite-1 having substantially uniform hexagonal column morphology and substantially free of crystal twinning

### BACKGROUND OF THE INVENTION

Molecular sieve materials, both natural and synthetic, have been demonstrated in the past to have catalytic properties for various types of hydrocarbon conversion. Molecular sieves that find application in catalysis include any of the naturally occurring or synthetic crystalline molecular sieves. Examples of these zeolites include large pore zeolites, intermediate pore size zeolites, and small pore zeolites. These zeolites and their isotypes are described in “Atlas of Zeolite Framework Types”, eds. W. H. Meier, D. H. Olson and Ch. Baerlocher, Elsevier, Fifth Edition, 2001. A large pore zeolite generally has a pore size of at least about 7 Å and includes LTL, VFI, MAZ, FAU, OFF, *BEA, and MOR framework type zeolites (IUPAC Commission of Zeolite Nomenclature). Examples of large pore zeolites include mazzite, offretite, zeolite L, VPI-5, zeolite Y, zeolite X, omega, and Beta. An intermediate pore size zeolite generally has a pore size from about 5 Å to less than about 7 Å and includes, for example, MFI, MEL, EUO, MTT, MFS, AEL, AFO, HEU, FER, MWW, and TON framework type zeolites (IUPAC Commission of Zeolite Nomenclature). Examples of intermediate pore size zeolites include ZSM-5, ZSM-11, ZSM-22, MCM-22, silicalite-1, and silicalite-2. A small pore size zeolite has a pore size from about 3 Å to less than about 5 Å and includes, for example, CHA, ERI, KFI, LEV, SOD, and LTA framework type zeolites (IUPAC Commission of Zeolite Nomenclature). Examples of small pore zeolites include ZK-4, ZSM-2, SAPO-34, SAPO-35, ZK-14, SAPO-42, ZK-21, ZK-22, ZK-5, ZK-20, zeolite A, chabazite, zeolite T, gmelinite, ALPO-17, and clinoptilolite.

ZSM-5 is an MFI-type zeolite, usually an aluminosilicate zeolite, which has been found useful as a catalyst in a variety of processes for preparing, converting, separating or purifying organic compounds. The earliest ZSM-5 zeolites were prepared using an organic template in the synthesis mixture which directed the formation of the ZSM-5 structure. Known ZSM-5 zeolites prepared using an organic template generally have a SiO₂/Al₂O₃ molar ratio of at least 60 and frequently considerably greater than 60 (see e.g. U.S. Pat. No. 4,797,267). So-called “inorganic” ZSM-5 zeolites, made in the absence of an organic template, were prepared in the 1980's. Typically these have a SiO₂/Al₂O₃ molar ratio of from 20 to about 30 to 40. SiO₂/Al₂O₃ ratios of up to 80 have been reported (e.g., Zeolites 1989 Vol. 9, 363-370).

The morphology of ZSM-5 crystals produced using an organic template can vary. For example, large elongated hexagonal prisms, whose corners may be rounded, was reported (see Studies in Surface Science and Catalysis 33, “Synthesis of High Silica Aluminosilicate Zeolites” (Elsevier), P. A. Jacobs and J. A. Martens). Crystals of ZSM-5 which are agglomerates of smaller, elementary hexagonal crystallites are also illustrated. In the presence of an extremely high proportion of silica (e.g., U.S. Pat. No. 4,797,267), ZSM-5 crystals may be rod shaped, i.e., elongated crystals with substantially parallel sides and blunt ends. The morphology of inorganic ZSM-5 crystals tends to be ellipsoidal (Zeolites 1989 Vol. 9, 363-370).

Researchers at Sogang University, Korea (e.g., J. Am. Chem. Soc., 2001, 123, 9769) reported synthesis of relative uniform ZSM-5. ZSM-5 with average crystal size of 2.0×1.5×0.8 μm³ (large ZSM-5) was synthesized from a gel with molar ratios of TEOS:TPAOH:NaAlO₂:H₂O=10:1:0.05:600, where TEOS and TPAOH represent tetraethylorthosilicate and tetrapropylammonium hydroxide, respectively, and the composition of NaAlO₂ was Na₂O=31-35% and Al₂O₃=34-39%. Also, ZSM-5 with average crystal size of 1.0×0.7×0.4 μm³ (medium ZSM-5) was synthesized from a gel with molar ratios of TEOS:TPAOH:NaAlO₂:H₂O=6:1:0.05:460 and ZSM-5 with average crystal size of 0.3×0.2×0.1 μm³ (small ZSM-5) was synthesized from a gel with molar ratios of TEOS:TPAOH:NaAlO₂:H₂O=7:1:0.06:280. The ZSM-5 crystals as reported by researchers at Sogang University have roughly the same shape and similar size.

However, the ZSM-5 zeolite disclosed by researchers at Sogang University has limited crystal size and low uniformity. It is well known that uniform crystal size and/or morphology of molecular sieves is often desirable to obtain superior performance in hydrocarbon conversion or separation processes, in film and coating applications and in macrostructure assembly. MFI crystals prepared following standard synthesis methods as disclosed in prior art have a broad size distribution and less discrete morphology, which may potentially limit their performance in the applications requiring uniformity of crystals. There is a need to develop molecular sieve or zeolite with high uniformity in both size and morphology.

The applicants have now identified a novel form of molecular sieve. The molecular sieve of the present invention have substantially uniform hexagonal column morphology. In particular, the crystals of this disclosure are substantially free of crystal twinning

### DESCRIPTION OF FIGURES

FIG. 1 is a SEM picture of the product of Example 6.

FIG. 2 is a SEM picture of the product of Example 1.

FIG. 3 is a SEM picture of the product of Example 7.

### SUMMARY OF THE INVENTION

This disclosure relates to a crystalline molecular sieve comprising silicalite-1 having substantially uniform hexagonal column morphology and substantially free of crystal twinning

In some embodiments, the crystal size of the crystalline molecular sieve is substantially uniform. In some aspects, the molecular sieve has a span of less than 3 measured by laser scattering.

In some aspects, the crystalline molecular sieve has less than 10% crystal twinning measured by SEM. In further embodiments, the size of the molecular sieve crystal is at least 0.1 um (micrometer) measured by laser scattering. In other aspects, the crystalline molecular sieve has a morphology uniformity of at least 95%.

In yet other embodiments, the crystalline molecular sieve has an edge/height ratio of the hexagonal column crystal in the range of 0.5 to 5.

In other embodiments this disclosure relates to a method of making a crystalline molecular sieve of this disclosure, the method comprises:
(a) providing a mixture comprising at least one source of at least one tetravalent element (Y), at least one source of hydroxide ion, at least one directing-agent (R), water, optionally at least one source of at least one metal element (M), the mixture having the following molar composition:
   H₂O/y=10 to 1000
   M/Y=0-0.5
   OH⁻/Y=0.41 to 0.74, preferably 0.45 to 0.6, more preferably 0.5;
   R/Y=0.001 to 2
      wherein R comprises at least one of TPAOH, TPACl, TPABr, TPAI, and TPAF, wherein OH⁻/Y is not corrected for trivalent ion;
(b) submitting the mixture at crystallization conditions to form a product comprising the crystalline molecular sieve, wherein the crystallization conditions comprise a temperature in the range of from 100° C. to 250° C., a crystallization time from about 1 hour to 200 hours; a heating rate in the range from at least 20° C./h, and a stirring speed at least 10 RPM; and
(c) recovering the crystalline molecular sieve.

In some embodiments, the tetravalent element is silicon. In other embodiments, the stirring speed is less than 600 RPM. In some preferred embodiments, the stirring speed is in the range of 50-350 RPM. In yet other aspects, the OH⁻/Y is in the range of 0.5-1.

In other embodiments this disclosure relates to a process for hydrocarbon conversion, comprising the step of contacting a hydrocarbon feedstock with the crystalline molecular sieve of this disclosure under conversion conditions to form a conversion product.

### DETAILED DESCRIPTION OF THE INVENTION

### Introduction

All patents, patent applications, test procedures, priority documents, articles, publications, manuals, and other documents cited herein are fully incorporated by reference to the extent such disclosure is not inconsistent with the present invention and for all jurisdictions in which such incorporation is permitted.

When numerical lower limits and numerical upper limits are listed herein, ranges from any lower limit to any upper limit are contemplated.

As used in this specification, the term “framework type” is used in the sense described in the “Atlas of Zeolite Framework Types,” 2001.

As used herein, the numbering scheme for the Periodic Table Groups is used as in Chemical and Engineering News, 63(5), 27 (1985).

The term “wppm” as used herein is defined as parts per million by weight.

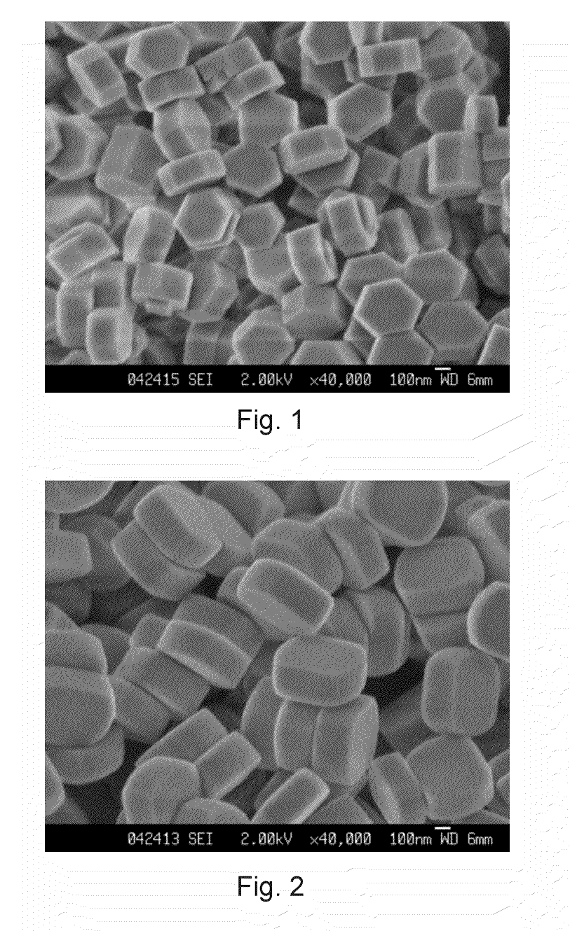

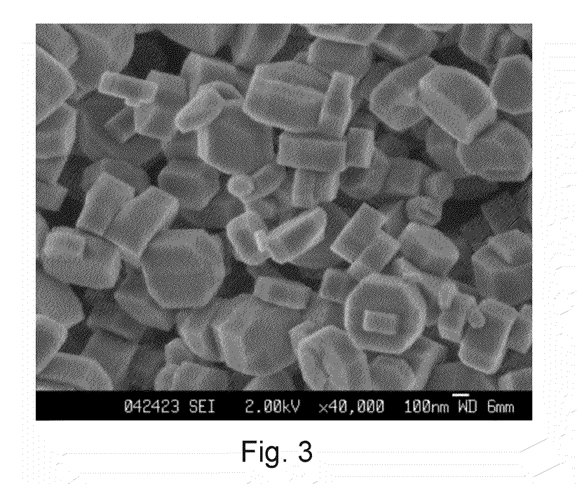

The crystal morphology and uniformity of crystal morphology can be measured by Scanning Electron Microscopy (SEM). A SEM picture having at least 50 visible crystals is used for this purpose. For example, in the case of FIG. 1, it is observed that at least 95% of the crystals have hexagonal column morphology. The term “substantially uniform hexagonal column morphology” as used herein means that at least 90% of the crystals in a SEM picture having at least 50 visible crystals have hexagonal column morphology.

The term “quasi parallelepiped morphology” (QPM) as used herein means a parallelepiped morphology having gently round-off ends. An example of an individual crystal having quasi parallelepiped morphology is illustrated in FIG. 2. The quasi parallelepiped morphology is further characterized by sharp edges of the crystal particle as shown in FIG. 2.

The term “hexagonal column morphology” (HEX) as used herein means a crystal morphology having hexagonal column shape. An example of an individual crystal having hexagonal column morphology is illustrated in FIG. 1.

When the average crystal size and crystal size distribution is measured by laser scattering, Gaussian-type particle size distribution curves are obtained. The average crystal size is expressed by D(50) in micrometer. The crystal size distribution and uniformity are expressed in D(10), D(50), D(90) and span. A D(c) number of, e.g., 1 micrometer, means that c% of the volume of the particles is smaller than 1 micrometer. The span is calculated as [D(90)-D(10)]/D(50) and indicates the width of the particle size distribution. In a case when the uniformity of crystal size is measured by laser scattering, the term “substantially uniform in size” as used herein means a span of 3 or less.

The term “substantially free of crystal twinning” as used herein means that less than 20% of the crystals in a SEM picture having at least 50 visible crystals are twinned crystals. A twinned crystal is defined as a crystal having 90-degree rotational intergrowth.

In some aspects, the crystalline molecular sieve composition of matter has an average molecular sieve crystal size of at least 0.1 micrometer, preferably at least 0.2 micrometer measured by SEM or laser scattering. In some embodiments, the crystalline molecular sieve composition of matter has an average molecular sieve crystal size of at least 1 micrometer.

In other embodiments of this disclosure, the uniformity of crystal size (measured by laser scattering) of the crystalline molecular sieve composition of matter has a span of 10 or less, preferably 8 or less, more preferably 6 or less, even more preferably 5 or less, yet even more preferably 4 or less, yet even more preferably 3 or less, and most preferably 2 or less.

In some embodiments of this disclosure, the crystalline molecular sieve composition of matter of this disclosure has a hexagonal column morphology uniformity of at least 90%, preferably at least 95%, based on a SEM picture with at least 50 visible crystals.

In some embodiments of this disclosure, the crystalline molecular sieve composition of matter of this disclosure has less than 20%, preferably less than 10% of crystal twinning, based on a SEM picture with at least 50 visible crystals.

### Formulation of the Hydrothermal Reaction Mixtures

Synthetic molecular sieves are often prepared from aqueous hydrothermal reaction mixtures (synthesis mixture(s) or synthetic gel(s)) comprising sources of appropriate oxides. Organic directing agents may also be included in the hydrothermal reaction mixture for the purpose of influencing the production of a molecular sieve having the desired structure. The use of such directing agents is discussed in an article by Lok et al. entitled “The Role of Organic Molecules in Molecular Sieve Synthesis” appearing in Zeolites, Vol. 3, October, 1983, pp. 282-291.

After the components of the hydrothermal reaction mixture are properly mixed with one another, the hydrothermal reaction mixture is subjected to appropriate crystallization conditions. Such conditions usually involve heating of the hydrothermal reaction mixture to an elevated temperature possibly with stirring. Room temperature aging of the hydrothermal reaction mixture is also desirable in some instances.

After the crystallization of the hydrothermal reaction mixture is complete, the crystalline product may be recovered from the remainder of the hydrothermal reaction mixture, especially the liquid contents thereof. Such recovery may involve filtering the crystals and washing these crystals with water. However, in order to remove the entire undesired residue of the hydrothermal reaction mixture from the crystals, it is often necessary to subject the crystals to a high temperature calcination e.g., at 500° C., possibly in the presence of oxygen. Such a calcination treatment not only removes water from the crystals, but this treatment also serves to decompose and/or oxidize the residue of the organic directing agent which may be occluded in the pores of the crystals, possibly occupying ion exchange sites therein.

The crystalline molecular sieve composition of matter of this disclosure may be prepared from a hydrothermal reaction mixture comprising at least one source of at least one tetravalent element (Y), at least one source of at least one trivalent element (X), at least one source of hydroxide ion, at least one directing-agent (R), water, and optionally sources of alkali or alkali earth metal (M), e.g., sodium, or potassium, and water, the hydrothermal reaction mixture having a composition, in terms of mole ratios, within the following ranges as shown in Table 1:

TABLE 1

| Reactants | Useful | Preferred | More preferred |
|---|---|---|---|
| Y/X₂ | 500-Infinity | 600-10000 | 800-5000 |
| H₂O/YO₂ | 10-1000 | 15-500 | 20-200 |
| OH⁻/YO₂* | 0.41-0.74 | 0.45-0.6 | 0.5 |
| M/YO₂ | 0-2 | 0-1 | 0-0.5 |
| R/YO₂ | 0.075-5 | 0.1-2 | 0.1-1 |

*The OH⁻/YO₂ is calculated without correction of trivalent element source.

The sources of the various elements required in the final product may be any of those in commercial use or described in the literature, as may the method of preparation of the synthesis mixture.

Y is a tetravalent element selected from Groups 4-14 of the Periodic Table of the Elements, such as silicon and/or germanium, preferably silicon. In some embodiments of this disclosure, the source of Y comprises solid YO₂, preferably about 30 wt % solid YO₂ in order to obtain the crystal product of this disclosure. When YO₂ is silica, the use of a silica source containing preferably about 30 wt % solid silica, e.g., silica sold by Degussa under the trade names Aerosil® or Ultrasil (a precipitated, spray dried silica containing about 90 wt % silica), an aqueous colloidal suspension of silica, for example one sold by Grace Davison under the trade name Ludox®, or HiSil® (a precipitated hydrated SiO₂ containing about 87 wt % silica, about 6 wt % free H₂O and about 4.5 wt % bound H₂O of hydration and having a particle size of about 0.02 micrometer) favors crystal formation from the above mixture. Preferably, therefore, the YO₂, e.g., silica, source contains about 30 wt % solid YO₂, e.g., silica, and more preferably about 40 wt % solid YO₂, e.g., silica. The source of silicon may also be a silicate, e.g., an alkali metal silicate, or a tetraalkylorthosilicate.

X is a trivalent element selected from Groups 3-13 of the Periodic Table of the Elements, such as aluminum, and/or boron, and/or iron and/or gallium, preferably aluminum. The source of X₂, e.g., aluminum, is mainly from the impurities of the Y source. Low level, e.g., less than 1000 wppm, of trivalent element compounds may exist in the sources of Y element.

The alkali or alkali earth metal element is advantageously lithium, sodium, potassium, calcium, or magnesium. The source of alkali or alkali earth metal element is advantageously being metal oxide, metal chloride, metal fluoride, metal sulfate, or metal nitrate. The sodium source advantageously being sodium hydroxide. The alkali metal may also be replaced by ammonium (NH₄⁺) or its equivalents, e.g., alkyl-ammonium ion.

Directing agent R comprises at least one of tetrapropylammonium salts, such as, tetrapropylammonium hydroxide (TPAOH), tetrapropylammonium chloride (TPACl), tetrapropylammonium bromide (TPABr), tetrapropylammonium iodide (TPAI), and tetrapropylammonium fluoride (TPAF).

The source of OH⁻ source is advantageously organic ammonium hydroxide, such as TPAOH, ammonium hydroxide, alkali metal oxide, e.g., Li₂O, Na₂O, K₂O, Rb₂O, Cs₂O, Fr₂O, or any combination thereof; alkali metal hydroxide, e.g., LiOH, NaOH, KOH, RbOH, CsOH, FrOH, or any combination thereof; ammonium hydroxide, alkali earth metal oxide, e.g., BeO, MgO, CaO, SrO, BaO, RaO, or any combination thereof; alkali earth metal hydroxide, e.g., Be(OH)₂, Mg(OH)₂, Ca(OH)₂, Sr(OH)₂, Ba(OH)₂, Ra(OH)₂, or any combination thereof; oxide(s) or hydroxide(s) of any element selected from Groups 3-17; and any combination thereof

The OH⁻/Y, e.g., OH⁻/Si molar ratio as used in this disclosure does not include correction of acid in the hydrothermal reaction mixture. It is calculated based on the total mole of hydroxide added to the hydrothermal reaction mixture.

In some embodiments of this disclosure, the OH⁻/Y molar ratio as used in this disclosure is in the range of from 0.41 to 0.74 and the R/Y, e.g., R/Si molar ratio as used in this disclosure is in the range of from 0.075 to 5. The following OH⁻/Y molar ratios are useful lower OH⁻/Y molar ratios limits: 0.41, 0.42, 0.43, 0.44, 0.45, 0.46, 0.47, 0.48, 0.49, 0.5, 0.55, 0.6, 0.7, 0.71, 0.72, and 0.73. The following OH⁻/Y molar ratios are useful upper OH⁻/Y molar ratios limits: 0.42, 0.43, 0.44, 0.45, 0.46, 0.47, 0.48, 0.49, 0.5, 0.55, 0.6, 0.7, 0.71, 0.72, 0.73, and 0.74. The OH⁻/Y molar ratio of a synthesis mixture useful for this disclosure ideally falls in a range between any one of the above-mentioned lower limits and any one of the above-mentioned upper limits, so long as the lower limit is less than or equal to the upper limit. The following R/Y molar ratios are useful lower R/Y molar ratios limits: 0.075, 0.08, 0.09, 0.1, 0.2, 0.3, 0.4, 0.5, and 1. The following R/Y molar ratios are useful upper R/Y molar ratios limits: 5, 2, 1.5, 1, 0.5, 0.4, 0.3, and 0.2. The R/Y molar ratio of a synthesis mixture useful for this disclosure ideally falls in a range between any one of the above-mentioned lower limits and any one of the above-mentioned upper limits, so long as the lower limit is less than or equal to the upper limit.

In some embodiments of this disclosure, the M/Y, e.g., M/Si molar ratio as used in this disclosure is in the range of from 0 to 2. In other embodiments, the M/Y molar ratio as used in this disclosure is in the range of from 0 to 1. In yet other embodiments, the M/Y molar ratio as used in this disclosure is in the range of from 0 to 0.5. The following M/Y molar ratios are useful lower M/Y molar ratios limits: 0, 0.001, 0.005, 0.01, 0.05, 0.1, 0.2, 0.3, 0.4, 0.5, and 1. The following M/Y molar ratios are useful upper M/Y molar ratios limits: 2, 1.5, 1, 0.5, 0.4, 0.3, and 0.2. The M/Y molar ratio of a synthesis mixture useful for this disclosure ideally falls in a range between any one of the above-mentioned lower limits and any one of the above-mentioned upper limits, so long as the lower limit is less than or equal to the upper limit.

In some embodiments of this disclosure, the H₂O/Y, e.g., H₂O/Si molar ratio as used in this disclosure is in the range of from 10 to 1000. In other embodiments, the H₂O/Y molar ratio as used in this disclosure is in the range of from 15 to 500. In yet other embodiments, the H₂O/Y molar ratio as used in this disclosure is in the range of from 20 to 200. The following H₂O/Y molar ratios are useful lower H₂O/Y molar ratios limits: 10, 15, 20, 25, 30, 50, 100, 200, and 500. The following H₂O/Y molar ratios are useful upper H₂O/Y molar ratios limits: 1000, 800, 600, 500, 400, 300, 200, 100, 90, 80, 70, 60, and 50. The H₂O/Y molar ratio of a synthesis mixture useful for this disclosure ideally falls in a range between any one of the above-mentioned lower limits and any one of the above-mentioned upper limits, so long as the lower limit is less than or equal to the upper limit.

In some embodiments of this disclosure, the Y/X₂, e.g., Si/Al₂, molar ratio as used in this disclosure is in the range of from 500 to infinity. In other embodiments, the Y/X₂ molar ratio as used in this disclosure is in the range of from 1000 to 10000. In yet other embodiments, the Y/X₂ molar ratio as used in this disclosure is in the range of from 1000 to 5000. The following Y/X₂ molar ratios are useful lower Y/X₂ molar ratios limits: 500, 600, 700, 800, 900, 1000, 1500, and 2000. The following Y / X₂ molar ratios are useful upper Y/X₂ molar ratios limits: infinity, 10000, 5000, 4000, 3000, 2000, 1000, 900, 800, 700, and 600. The Y/X₂ molar ratio of a synthesis mixture useful for this disclosure ideally falls in a range between any one of the above-mentioned lower limits and any one of the above-mentioned upper limits, so long as the lower limit is less than or equal to the upper limit.

It should be realized that the hydrothermal reaction mixture components can be supplied by more than one source. The hydrothermal reaction mixture can be prepared either batchwise or continuously. Crystal size and crystallization time of the silicalite-1 of this disclosure may vary with the nature of the hydrothermal reaction mixture employed and the crystallization conditions.

It will be understood by a person skilled in the art that the synthesis mixture having a composition within the molar ranges as discussed above means that the synthesis mixture is the product of mixing, adding, reacting, or by any means of providing such a mixture, wherein such product has a composition within the molar ranges as discussed above. The product of mixing, adding, reacting, or by any means of providing such a mixture may or may not contain individual ingredients when the synthesis mixture was prepared. The product of mixing, adding, reacting, or by any means of providing such a mixture, may even contain reaction product of individual ingredients when the synthesis mixture was prepared by mixing, adding, reacting, or by any means of providing such a mixture.

Optionally the hydrothermal reaction mixture may contain seed crystals. It is well known that seeding a molecular sieve synthesis mixture frequently has beneficial effects, for example in controlling the particle size of the product, avoiding the need for an organic template, accelerating synthesis, and improving the proportion of product that is of the intended framework type. In some embodiments of this disclosure, the synthesis of the crystalline molecular sieve is facilitated by the presence of 0 to about 25 wt %, preferably about 1 to about 5 wt %, seed crystals based on total weight of tetrahedral element oxide (e.g., silica) of the hydrothermal reaction mixture.

### Crystallization Conditions

The hydrothermal reaction of this disclosure is carried with agitation. The rate of the agitation is measured by the rotation speed of the stirrer in rotation per minute (RPM), by the tip-speed in m/s, or by volume average stirring speed. If a stirrer having a diameter of m (meter) is rotated with n RPM, then the tip speed is calculated as π*m*n/60 (m/s). In the vessels used in this disclosure, a stirring rate of 100 RPM corresponds to a tip speed of 0.146 m/s. The effect of stirring may also expressed as volume average speed which is calculated as π*m*n/180 (m/s).

The mixture is crystallized under crystallization conditions comprising a temperature in the range of from 100° C. to 250° C., preferably 120° C. to 200° C., a crystallization time from about 1 hour to 1000 hours, preferably from about 5 hours to about 200 hours, more preferably from about 10 hours to about 100 hours; a heating rate in the range from at least 10° C./h, preferably at least 20° C./h, and most preferably at least 50° C./h, and a stirring speed at least 5 RPM, preferably at least 10 RPM, more preferably at least 50 RPM, even more preferably at least 100 RPM, yet even more preferably at least 150 RPM, still yet more preferably at least 200 RPM, still yet more preferably at least 250 RPM, still yet more preferably at least 300 RPM, and most preferably 350 RPM, or a stirring speed at least 0.005 m/s, preferably at least 0.01 m/s, more preferably at least 0.05 m/s, even more preferably at least 0.1 m/s, yet even more preferably at least 0.15 m/s, still yet more preferably at least 0.2 m/s, still yet more preferably at least 0.25 m/s, still yet more preferably at least 0.5 m/s, and most preferably 1 m/s, or a volume average stirring speed at least 0.0016 m/s, preferably at least 0.0033 m/s, more preferably at least 0.016 m/s, even more preferably at least 0.033 m/s, yet even more preferably at least 0.05 m/s, still yet more preferably at least 0.067 m/s, still yet more preferably at least 0.0833 m/s, still yet more preferably at least 0.167 m/s, and most preferably 0.333 m/s.

In some embodiments, the crystallization conditions comprises a combination of a heating rate at least 10° C./h and a stirring speed at least 250 RPM or a tip speed at least 0.146 m/s. In other embodiments, the crystallization conditions comprises a combination of a heating rate at least 50° C./h and a stirring speed at least 100 RPM or a tip speed at least 0.100 m/s. In yet other embodiments, the crystallization conditions comprises a combination of a heating rate at least 100° C./h and a stirring speed at least 50 RPM or a tip speed at least 0.050 m/s.

All heating rate as used herein means heating a mixture continuously for at least 5 minutes, preferably at least 10 minutes, at the specified heating rate. All stirring speed as used herein means stirring a mixture continuously for at least 5 minutes, preferably at least 10 minutes, at the specified stirring speed.

The procedure may optionally include an aging period, either at room temperature (˜25° C.) or at a moderately elevated temperature (less than 100° C.), with or without agitation, before the hydrothermal treatment (“hydrothermal reaction”) at more elevated temperature. The aging period may last from 0 to 500 hours, preferably from 1 to 100 hours, more preferably from 2 to 50 hours, and most preferably from 5 to 20 hours.

The crystalline molecular sieve from the synthesis may further be filtrated, washed with water, and/or dried. The crystalline molecular sieve of this disclosure formed by crystallization may be recovered and subjected for further treatment, such as, ion-exchange with ammonium salt(s) (e.g., ammonium hydroxide, ammonium nitrate, ammonium chloride, ammonium sulfate, ammonium phosphate, or any combination thereof), impregnation, dealumination, and/or calcination in an oxidative atmosphere (e.g., air, gas with an oxygen partial pressure of greater than 0 kPa-a) at a temperature of greater than 200° C., preferably at least 300° C., more preferably at least 400° C., and most preferably at least 500° C.

Impregnation allows deposition of metal salts on the molecular sieve, e.g. salts of noble metals. Dealumination can be done, e.g., by steaming or by any chemical treatment. These treatments result in modifying the molecular sieve framework composition.

The molecular sieve may be bound with a matrix material such as clay or silica to increase the physical strength of the material for its use as a catalyst in a variety of processes.

### INDUSTRIAL APPLICATIONS

The crystalline molecular sieve of this disclosure is useful in the production and conversion of organic compounds, for example cracking, hydrocracking, dewaxing, isomerization (including e.g. olefin summarization and skeletal summarization e.g. of butane), oligomerization, trimerization, polymerization, alkylation, dealkylation, hydrogenation, dehydrogenation, dehydration, cyclization and aromatization. The present invention therefore provides a process for the production or conversion of an organic compound comprising the use of a catalyst of the molecular sieve described above. The molecular sieve can also be used (either as initially prepared or in a modified form) in a selective adsorption process e.g. a separation or purification.

These and other facets of the present invention are exemplified by the following Examples.

### EXAMPLES

In the Examples, the XRD diffraction patterns of the as-synthesized materials were recorded on a STOE Stadi-P Combi transmission XRD using copper Kα radiation in the 20 range of 2 to 40 degrees.

The SEM images were obtained on a JEOL JSM-6340F Field-Emission-Gun scanning electron microscope is used, operating at 2 kV and 12 μA. The crystal size was measured by averaging the size of multiple crystals as shown in the SEM.

The particle size analysis is performed using a Mastersizer APA2000, from Malvern Instruments Limited, equipped with a 4 mW laser beam, based on laser scattering by randomly moving particles in a liquid medium. The samples to be measured are dispersed in water and sonicated in situ to ensure proper dispersion.

Autoclaves with a capacity of 30 mL were used for the synthesis. These autoclaves were equipped with heating jacket, internal thermocouples, and mechanical stir.

The following Table 2 lists all chemicals, raw materials, and their sources used in the examples of this disclosure.

TABLE 2

| Name | Composition | Source |
|---|---|---|
| Tetrapropylammonium hydroxide (TPAOH) | 20 wt % in water | Aldrich |
| Tetraethylorthosilicate (TEOS) | 98% | Aldrich |
| NaOH | 98% | Aldrich |
| Al(OH)₃ | 96+% | Aldrich |

### Examples 1-6

Examples 1-6 were prepared with the following procedure:
(a) TEOS was added to aqueous TPAOH under stirring;
(b) the mixture of step (a) was stirred for 1 hour at 25° C.;
(c) water was added to the mixture of step (b);
(d) the mixture of step (c) was stirred at 25° C. for 12 hours; then
(e) the mixture of step (d) was transferred to a stirred autoclave;
(f) the autoclave was heated to 160° C. with 100° C./h heating rate (in Example 3 a heating rate of 20° C./h was used);
(g) the autoclave was maintained at 160° C. for 40 hours with 350 RPM stirring rate;
(h) the autoclave was cooled down to 25° C. and the crystals were recovered and washed using centrifugation or filtration, and dried at 60° C. for 24 hours; and
(i) the product of step (h) was analyzed by XRD, laser scatting, and SEM.

The following Table 3 summarized the slurry composition and product characterization results.

TABLE 3

| Example | 1 | 2 | 3 | 4 | 5 | 6 |
|---|---|---|---|---|---|---|
| Al/Si (molar) | 0 | 0 | 0 | 0 | 0 | 0 |
| TPA/Si (molar) | 0.2 | 0.4 | 0.5 | 0.5 | 0.5 | 0.5 |
| H₂O/Si (molar) | 50 | 50 | 50 | 30 | 40 | 50 |
| M/Si (molar) | 0 | 0 | 0 | 0 | 0 | 0 |
| OH⁻/Si (molar) | 0.3 | 0.4 | 0.5 | 0.5 | 0.5 | 0.5 |
| XRD | MFI | MFI | MFI | MFI | MFI | MFI |
| D50 (micron) | 0.5 | 0.2 | 0.7 | 0.7 | 0.4 | 0.4 |
| Span | 1.0 | 2.1 | 1.6 | 1.9 | 1.8 | 1.8 |
| Morphology | QPM | QPM | HEX | HEX | HEX | HEX |
| Morphology uniformity | 95% | 95% | 95% | 95% | 95% | 95% |
| Twinning | 2% | 2% | 10% | 10% | 10% | 10% |

### Examples 7-9

Examples 7-9 were prepared with the following procedure:
(a) TEOS was added to aqueous TPAOH under stirring;
(b) the mixture of step (a) was stirred for 1 hour at 25° C.;
(c) an aqueous solution composed of 10 wt % NaOH and 7.8 wt % Al(OH)₃ was added slowly under stirring;
(d) water was added to the mixture of step (c);
(e) the mixture of step (d) was stirred at 25° C. for 12 hours; then
(f) the mixture of step (e) was transferred to a stirred autoclave;
(g) the autoclave was heated to 160° C. with 100° C./h heating rate;
(h) the autoclave was maintained at 160° C. for 40 hours with 350 RPM stirring rate;
(i) the autoclave was cooled down to 25° C. and the crystals were recovered and washed using centrifugation or filtration, and dried at 60° C. for 24 hours; and
(j) the product of step (i) was analyzed by XRD, laser scatting, and SEM.

The following Table 4 summarized the slurry composition and product characterization results.

TABLE 4

| Example | 7 | 8 | 9 |
|---|---|---|---|
| Al/Si (molar) | 0.004 | 0.01 | 0 |
| TPA/Si (molar) | 0.5 | 0.5 | 0.75 |
| H₂O/Si (molar) | 50 | 50 | 50 |
| M/Si (molar) | 0.01 | 0.025 | 0 |
| OH⁻/Si (molar) | 0.51 | 0.525 | 0.75 |
| XRD | MFI | MFI | MFI |
| D50 (micron) | 0.9 | 1.4 | 11.5 |
| Span | 412.8 | 1.6 | 1.8 |
| Morphology | non-uniform HEX | undefined | undefined |
| Morphology uniformity | NA | NA | NA |
| Twinning | NA | NA | NA |

### Examples 10-12

Examples 10-12 were prepared with the following procedure:
(a) TEOS was added to aqueous TPAOH under stirring;
(b) the mixture of step (a) was stirred for 1 hour at 25° C.;
(c) water was added to the mixture of step (b);
(d) the mixture of step (c) was stirred at 25° C. for 12 hours; then
(e) the mixture of step (d) was transferred to a stirred autoclave;
(f) the autoclave was heated to 160° C. with 100° C./h heating rate;
(g) the autoclave was maintained at 160° C. for 40 hours with 350 RPM stirring rate;
(h) the autoclave was cooled down to 25° C. and the crystals were recovered and washed using centrifugation or filtration, and dried at 60° C. for 24 hours; and
(i) the product of step (h) was analyzed by XRD, laser scatting, and SEM.

The following Table 5 summarized the slurry composition and product characterization results.

TABLE 5

| Example | 10 | 11 | 12 |
|---|---|---|---|
| Al/Si (molar) | 0 | 0 | 0 |
| TPA/Si (molar) | 1 | 0.5 | 0.5 |
| H₂O/Si (molar) | 46 | 60 | 80 |
| M/Si (molar) | 0 | 0 | 0 |
| OH⁻/Si (molar) | 1 | 0.5 | 0.5 |
| XRD | MFI | MFI | MFI |
| D50 (micron) | 16.1 | 0.4 | 0.4 |
| Span | 1.4 | 2.2 | 1.9 |
| Morphology | undefined | HEX | HEX |
| Morphology uniformity | NA | 95% | 95% |
| Twinning | NA | 10% | 10% |

While the present invention has been described and illustrated by reference to particular embodiments, those of ordinary skill in the art will appreciate that the invention lends itself to many different variations not illustrated herein. For these reasons, then, reference should be made solely to the appended claims for purposes of determining the scope of the present invention.

1. A crystalline molecular sieve comprising silicalite-1 having a hexagonal column crystal with morphology uniformity of at least 90% as measured by SEM and having less than 20% crystal twinning as measured by SEM.

2. The crystalline molecular sieve of claim 1, wherein the crystal size of said crystalline molecular sieve has a span of 10 or less as measured by laser scattering.

3. The crystalline molecular sieve of claim 2, wherein said crystalline molecular sieve has less than 10% crystal twinning as measured by SEM.

4. The crystalline molecular sieve of claim 1, wherein the size of the molecular sieve crystal is at least 0.1 micrometer as measured by laser scattering.

5. The crystalline molecular sieve of claim 4, wherein said crystalline molecular sieve has a hexagonal column morphology uniformity of at least 95% as measured by SEM.

6. The crystalline molecular sieve of claim 1, wherein said crystalline molecular sieve has an edge/height ratio of the said hexagonal column crystal in the range of 0.5 to 5 as measured by SEM.

7. The molecular sieve of claim 1, wherein said crystalline molecular sieve has a span of less than 3 as measured by laser scattering.

8. A method of making a crystalline molecular sieve of claim 1 comprising the steps of:
(a) providing a mixture comprising at least one source of at least one tetravalent element (Y), at least one source of hydroxide ion, at least one directing-agent (R), water, said mixture having the following molar composition:
H₂O/Y=10 to 1000
OH⁻/Y=0.41 to 0.74
R/Y=0.001 to 2
wherein R comprises at least one of TPAOH, TPACl, TPABr, TPAI, and TPAF,
wherein OH⁻/Y is not corrected for trivalent ion;
(b) subjecting said mixture to crystallization conditions to form a product comprising said crystalline molecular sieve, wherein said crystallization conditions comprise a temperature in the range of from 100° C. to 250° C., a crystallization time from about 1 hour to 200 hours, a heating rate in the range from at least 20° C./h, and a stirring speed of at least 10 RPM; and
(c) recovering said crystalline molecular sieve.

9. The method of claim 8, wherein said mixture of step (a) further comprising at least one source of at least one metal element (M), wherein a molar ratio of M/Y is in the range from 0 to 0.5.

10. The method of claim 9, wherein said stirring speed is less than 600 RPM.

11. The method of claim 10, wherein said stirring speed is in the range of 50-350 RPM.

12. The method of claim 8, wherein said OH⁻/Y is in the range of 0.45-0.6.

13. The method of claim 12, wherein the said OH⁻/Y is 0.5.

14. The method of claim 13, wherein said tetravalent element is silicon.

15. A process for hydrocarbon conversion, comprising the step of:
contacting a hydrocarbon feedstock with said crystalline silicalite-1 molecular sieve recited in claim 1, under conversion conditions to form a conversion product.
